(19) United States
(12) Patent Application Publication
(10) Pub. No.: US 2006/0252143 A1
(43) Pub. Date: Nov. 9, 2006

(54) High resolution semiconductor bio-chip with configuration sensing flexibility

(75) Inventor: Wai Lo, Lake Oswego, OR (US)

Correspondence Address: LSI LOGIC CORPORATION, 1621 BARBER LANE, MS: D-106, MILPITAS, CA, 95035, US

(73) Assignee: LSI Logic Corporation

(21) Appl. No.: 11/122,356

(22) Filed: May 5, 2005

(51) Int. Cl. G06F 19/00; C12M 1/34

(52) U.S. Cl. 435/287.1; 702/19; 977/900

(57) ABSTRACT

Nano-tube/nano-fiber electrodes are integrated with electronic devices to form a single-chip nano-bio-sensor. The single-chip nano-bio-sensor which uses nano-meter scale electronic devices, includes sensing transistors in close proximity to nano-tube/nano-fiber electrodes, and provides an arrangement of the nano-tube/nano-fiber electrodes into high density clusters and groups so that sensitive, low noise detection of the activities of small cells, large cells and a network of cells is possible. The integrated, single-chip approach provides that differential signal extraction is possible. The single-chip nano-bio-sensor includes small feature size transistors. As such, only low supply voltage is needed.

### BACKGROUND

The present invention generally relates to semiconductor bio-chips, and more specifically relates to a single-chip nano-bio-sensor which includes nano-tube/nano-fiber electrodes with electronic devices.

The capability to probe into the activities of cells is of extreme importance. Monitoring the electrical activities of individual cells using electrode arrays, for example, has proved to be useful for understanding the physiology of cells as well as the functioning of neurons. The hybrid systems formed between the cells and the monitoring devices are also powerful systems for screening the effects of pharmaceuticals, assisting drug discoveries, categorizing stimulants and identifying environmental agents such as contaminants and biological warfare substances. Given proper system design, and the incorporation of suitable materials, hybrid systems capable of collecting information on cell networks will certainly lead to breakthroughs in medical diagnosis, drug development and other related scientific disciplines.

There are stringent requirements on the features of such devices if the above-mentioned goals are to be achieved. These include:
1. Biocompatibility: Any components in contact with cells should not result in the abnormal behavior or even death of the cells.
2. Size of the electrodes: The size of cells in human bodies as well as in most bacteria and non-vertebrates normally ranges from 1-100 μm. To collect information of the cells and cell networks, it is necessary to use electrodes with dimensions <<1 μm so that the response on specific locations of each cell (e.g., the axon of a neuron) can be recorded. Ideally, the size of the probe should be ≦100 nm in diameter. This is especially important in monitoring the activities of networks of cells, which will reveal the communication and coordination between them. Such information will be useful for understanding the functioning of complex neural networks.
3. Density of the electrode arrays: To ensure the collection of information from each cell, or even generate maps of activities of large cells and cell networks, the average separation between electrodes must be comparable to the size of the smallest cells, i.e., about 1 μm. This implies that high density of electrodes are needed.
4. Two-dimensional sensing arrays: The amount of information which can be collected will be limited, if the electrodes cannot be arranged in a 2-dimensional (2-D) fashion. A 2-D arrangement of the electrodes, however, cannot be achieved unless sufficient layers of wires for the electrodes and the supporting electronic devices are used for routing the signals.
5. Locations of the electrodes: Another critical factor in generating activity maps from individual cells and cell networks is the ability to select and manipulate the locations of the electrodes. This will impose a limitation on the fabrication techniques which are usable.
6. Electrode reliability: It is known that certain electrode materials which are available commercially, such as Au and Pt, can degrade and generate toxins after repeated use. Even without the degradation problems, it would not be possible to repeatedly fabricate electrodes with the desirable dimensions, e.g., 100 nm, and conveniently manipulate their locations.
7. Multiplexing of signal scores: With the large number of electrodes present in a 2-D fashion, it is critical to be able to recognize the source of each signal, i.e., multiplexing of the signals. Hence, any integrated bio-sensor should contain switching circuits for this purpose.
8. Noise reduction: The voltage generated by a neuron pulse is normally on the order of 1 mV, which is quite low and is hence susceptible to interference from different noise sources. It is necessary to maximize signal-to-noise levels through both system design and materials selection, i.e., maximize the conductivity of the electrodes and minimize system parasitics.
9. Information extraction: For some applications, such as exploring the propagation of electrical signals in cells, it would be more advantageous to read the relative changes in signals between selected electrodes. A differential signal monitoring of cell activities is therefore necessary for these applications. In addition, this mode of information extraction will also help to increase signal-to-noise levels.
10. Power consumption: Low power consumption is important for portable devices, especially implantable ones. Implantable devices can provide in vivo information of the cell activities that cannot be extracted in other environments. Low power consumption can be achieved by reducing supply voltage, and hence requires small feature size electronic devices, as well as reducing the parasitics of the circuitries.
11. Configuration versatility: Due to the large varieties of conditions in living organisms (e.g., cell size, network complexity), the systems would be much more useful if they can be configured efficiently, even only at certain steps of the design process, for each specific application. This is possible only if the fabrication of the electrodes can be fully manipulated and the systems assume certain configuration flexibility.

Different devices have been developed over the years either for monitoring cell activities or as hybrid bio-sensors. These are described separately below:
1. Conventional devices: These devices consist of relatively large electrodes (e.g. 10-500 μm in diameter) that are connected to electronic meters for recording cell activities.
2. Integrated devices: There are publications of sophisticated devices that integrated some of the electronic components needed for extracting the electronic signals generated by cell activities with certain electrodes such as Au and Pt. The size of the electrodes are typically 1-5 μm and the pitch of the electrodes is close to 10 μm. This integrated device approach has the advantage of minimizing the parasitics of the monitoring electronic circuits so that signal-to-noise level can be raised. It also has the advantage of being portable, or even implantable into living organisms.
3. Nano-tube/nano-fiber electrodes: Nano-tubes/nano-fibers of conducting materials such as C and ZnO were proposed as a refinement of the size of the electrodes. The diameter of these electrodes is typically 10-100 nm, which is potentially capable of resolving the intra-cell activities. In addition, these electrodes were grown on contact pads ranging from 0.2 μm to 20 μm which were deposited directly on solid, un-patterned substrates such as Si or SiO₂. The contact pads were connected to monitoring electronic devices separated from the electrode substrates, through metal wires deposited on the same substrates and between the contact pads. With the large number of electrodes on a single contact pad, the overall reliability of the electrodes is greatly enhanced.

The disadvantages of the first kind of devices are clear. Even if there is no bio-compatibility problem, the size of the electrodes, difficulties in fabricating and operating 2-D arrays, and the lack of portability all limited the usefulness of these devices.

As mentioned above, the main advantage of the second type of devices is their integration approach. Comparing the reported features of such devices with the requirements listed before, however, shows their shortcomings. The size of the electrodes, for example, cannot resolve the intra-cellular activities of most cells, nor can they give reliable information about the interactions between cells. Rather, they are more suitable for recording the collective activities of groups of cells. Another critical problem of these devices is that they are fabricated using CMOS devices with large feature sizes, e.g., a gate length of 0.5 μm, and operated using relatively high voltages, i.e., 5V. This, on the one hand, is consistent with the low density of the electrodes and, on the other hand, raises power consumption as well as reduces portability of the devices. The capability of these devices are therefore limited.

The third type of devices exploit the small dimensions, bio-compatibility and reliability of nano-tube/nano-fiber materials and use them as electrodes. However, the lack of integration between the electrodes and the electronic devices as well as the 2-D planar structured contact pads and wires will certainly result in low electrode density, high parasitic effects and poor signal-to-noise parameters.

Further, none of all these devices even considers differential extraction of cell activities.

### OBJECTS AND SUMMARY

An object of an embodiment of the present invention is to provide a single-chip nano-bio-sensor which integrates nano-tube/nano-fiber electrodes with electronic devices.

Another object of an embodiment of the present invention is to provide a single-chip nano-bio-sensor which uses nano-meter scale electronic devices, includes sensing transistors in close proximity to nano-tube/nano-fiber electrodes, and which provides an arrangement of nano-tube/nano-fiber electrodes in high density clusters and groups so that sensitive, low noise detection of the activities of small cells, large cells and network of cells is possible.

Yet another object of an embodiment of the present invention is to provide an integrated, single-chip approach, which provides that differential signal extraction is possible.

Still another object of an embodiment of the present invention is to provide a single-chip nano-bio-sensor which includes small feature size transistors, such that only low supply voltage is needed.

Briefly, and in accordance with at least one of the foregoing objects, an embodiment of the present invention provides a single-chip nano-bio-sensor which includes electronic devices on a chip, and nano-tube/nano-fiber electrodes on the chip, connected to the electronic devices. The nano-tube/nano-fiber electrodes are configured to monitor cell activity and provide signals to the electronic devices. The electronic devices are configured to process the signals which are received from the nano-tube/nano-fiber electrodes.

The nano-tube/nano-fiber electrodes are disposed on a signal acquisition module. The electronic devices further include a decoder, reference sensors, an amplifier and a multiplexer. The signal acquisition module and reference sensors are configured to provide signals to the amplifier, and the amplifier is configured to provide amplified signals to the multiplexer. The electronic devices further include a signal processing unit and a communication module. The multiplexer is configured to process signals received from the amplifier and provide signals to the signal processing unit which further processes the signals and provides signals to the communication module. The communication module is configured to communicate information about the cell activity which was monitored by the nano-tube/nano-fiber electrodes, to a destination outside the chip. A power supply is connected to the signal acquisition module, amplifier, reference sensors, multiplexer, signal processing unit and communication module, and ESD protection circuitry is provided.

The signal acquisition module includes threshold signal determining, clocking, signal routing and amplification circuitries between electrode pads and under an electrode layer, as well as groups of nano-tube/nano-fiber electrodes and signal routing wirings under the electrodes. Each nano-tube/nano-fiber electrode includes an electrode cluster which is disposed over, and connected to, a transistor.

### BRIEF DESCRIPTION OF THE DRAWINGS

The organization and manner of the structure and operation of the invention, together with further objects and advantages thereof, may best be understood by reference to the following description, taken in connection with the accompanying drawing, wherein:

FIG. 1 is a schematic diagram which illustrates a possible architecture of a single-chip nano-bio-sensor which is in accordance with an embodiment of the present invention;

FIG. 2 illustrates a possible layout of the signal acquisition module of the single-chip nano-bio-sensor which is shown in FIG. 1;

FIG. 3 illustrates a possible layout of each group of nano-tube/nano-fiber electrodes and supporting circuitries; and

FIG. 4 provides a cross-section of a possible physical structure of an electrode group which can be utilized in association with an embodiment of the present invention.

### DESCRIPTION

While the invention may be susceptible to embodiment in different forms, there are shown in the drawings, and herein will be described in detail, specific embodiments of the invention. The present disclosure is to be considered an example of the principles of the invention, and is not intended to limit the invention to that which is illustrated and described herein.

The present invention targets the fabrication of high performance, single-chip bio-sensors capable of monitoring sensitively the activities of cells and cell networks by integrating nano-tube/nano-fiber electrodes, nano-meter size CMOS devices and signal extraction/processing/transmission circuitries on to a single chip. The schematic diagram in FIG. 1 illustrates an example of a possible system architecture. As shown in FIG. 1, the architecture may include (but may not be limited to) the following structures provided on a single chip 10: a signal acquisition module 12, a decoder 14, an amplifier 16, a multiplexer 18, a signal processing unit 20, a communication module 22, a power supply 24, reference sensors 26 and an ESD protection unit 28. The amplifier 16 is configured to amplify the signals received from the signal acquisition module 12 and reference sensors 26, and provide the amplified signals to the multiplexer 18 which processes the signals and provides them to the signal processing unit 20. The signal processing unit 20 processes the signals and communicates with the communication module 22. As shown in FIG. 1, preferably the ESD protection unit 28 is configured to protect all components (i.e., the signal acquisition module 12, decoder 14, amplifier 16, multiplexer 18, signal processing unit 20, reference sensors 26 and communication module 22).

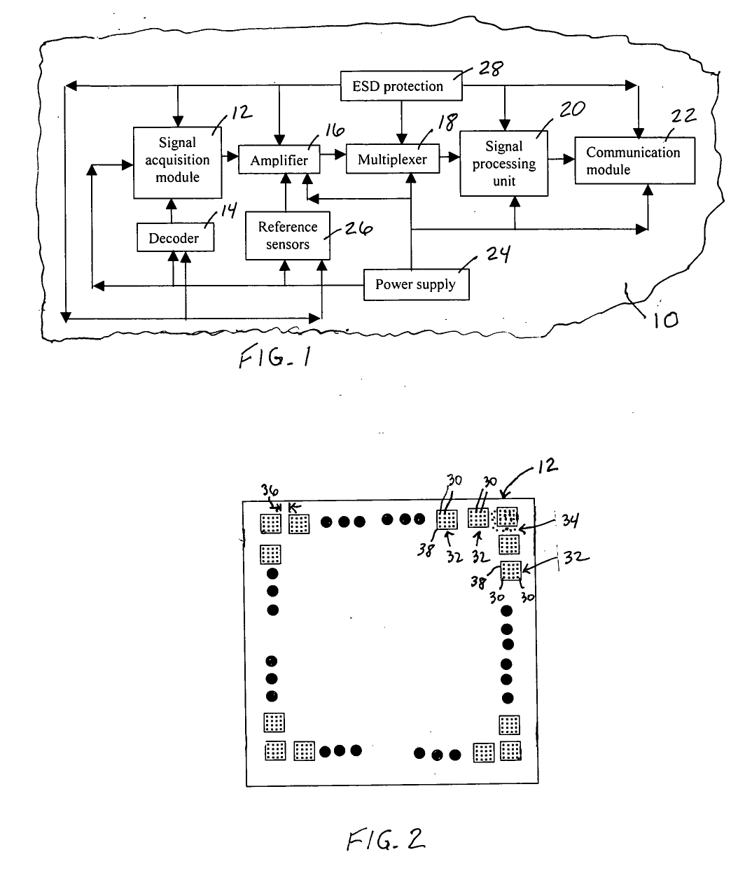

A possible layout of the signal acquisition module 12 is illustrated in FIG. 2. As shown, the signal acquisition module 12 consists of nano-tube/nano-fiber electrodes 30 arranged in groups 32, and threshold signal determining, clocking, switching and signal routing as well as preliminary amplification circuitries (identified with reference numeral 34). Specifically, the electrodes 30 are arranged in groups 32 on top of some electronic devices, such as CMOS transistors, that are configured to measure the activities of the cells in contact with the electrodes. As such, the CMOS transistor can be said to be data acquisition devices. In addition to electronic devices, there is also signal routing wiring which is disposed under the electrodes. The number and dimensions of the electrodes are adjustable. In addition, the total number of electrode groups, per row and column, are adjustable, as is the distance (i.e., dimension 36 in FIG. 2) between each electrode group 32. The supporting electronic circuitries 34, e.g., switches and amplifiers, are located in the areas between the electrode groups and under the electrode layer 38.

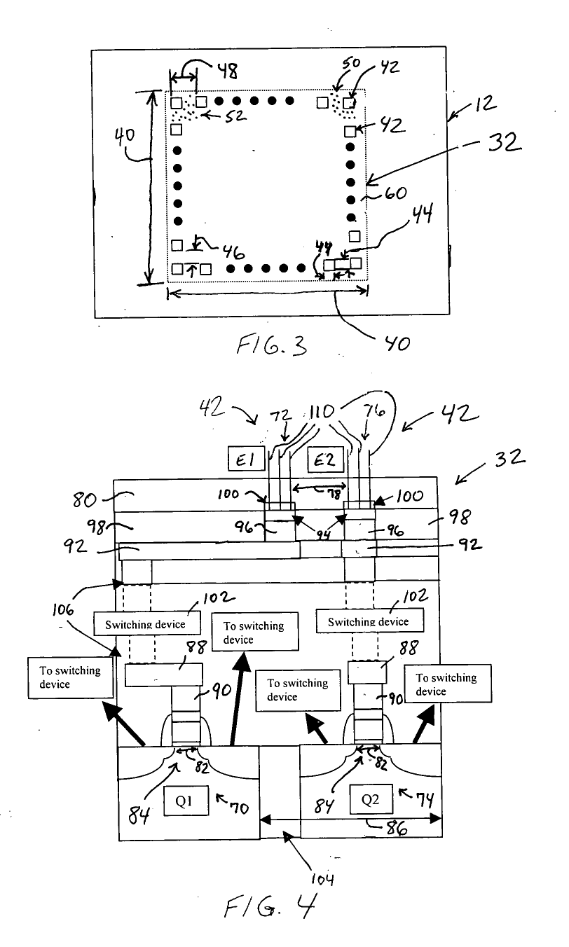

A possible layout of each electrode group 32 is further illustrated in FIG. 3. Each electrode group may have dimensions 40 of 5-50 μm (i.e., if the group is arranged as a square, the sides of the square may be between 5 and 50 μm wide) and may consist of a large number of nano-tube/nano-fiber electrode clusters 42 which have dimensions 44 of ˜100 nm (i.e., if the cluster 42 is arranged as a square, the sides of the square may be ˜100 nm wide). The dimension 44 of each electrode group and the separation (i.e., dimension 46 in FIG. 3) between the electrode clusters 42 are adjustable. However, in order to have high spatial resolution of measurements, the separation 46 between electrode clusters 42 should be ˜100 nm, i.e., pitch (i.e., dimension 48 in FIG. 3) of electrode clusters ˜200 nm. Data acquisition devices (e.g., CMOS) and signal routing wirings are provided under and around the electrodes (represented in FIG. 3 with reference numeral 50). Additionally, signal routing and preliminary amplification circuitries (represented in FIG. 3 with reference numeral 52) are provided between the electrode pads and under the electrode layer (i.e., in the substrate 60).

An electrode cluster pitch (i.e., dimension 48 in FIG. 3) of 200 nm is smaller than any advanced CMOS transistor, e.g., gate length <70 nm. Suppose the electrical signal (voltage or current) detectable by each electrode cluster 42 is recorded by a single transistor, packing the transistors (which have a pitch of 1 μm) under the electrode clusters 42 implies that some of the transistors will be accommodated in areas outside the electrode group. Communications between the transistors and the electrode clusters can be performed using conducting wires (either metals or other materials such as nano-tubes/nano-fibers).

The layers of conducting wires are also needed for addressing the sensing transistors (i.e., for determining which transistor is sensing the signal) using devices like switches, as well as for connecting the rest of the on-chip electronic devices. The switching devices are preferably located between the electrode groups. Together with the switching devices are preliminary amplifiers, such as differential amplifiers and Darlington amplifiers, for raising the signal levels before feeding them to the rest of the chip. This is particularly important for differential extraction of cell activities, as described earlier.

Another important implication of this approach of lay-out and integration is the programmability of the chip design. For example, with careful design, it is possible to manufacture in volume the chip with standard layouts and configure the interconnects between the transistors and the locations of the electrodes so as to customize the cell activities detection locations, signal processing and output formats, according to application specifications. This is in line with the RapidChip methodology of LSI Logic.

FIG. 4 illustrates a portion of the possible physical structure of an electrode group 32. In FIG. 4, reference numeral 70 identifies a transistor (“Q1”) for recording signals from nano-tube/nano-fiber electrode cluster (“E1”) 72, and reference numeral 74 identifies a transistor (“Q2”) for recording signals from nano-tube/nano-fiber electrode cluster (“E1”) 76. The electrode clusters E1 and E2 are preferably spaced about 100 nm apart (i.e., dimension 78 in FIG. 4), and extend from an electrode anchorage layer 80. Reference numeral 82 identifies the length of each of the gates 84 of the transistors Q1, Q2, which is preferably <70 nm. Reference numeral 86 identifies the transfer pitch which is preferably ˜1 μm. Reference numeral 88 identifies a wire layer (i.e., metal or other conductors) of each of the transistors Q1, Q2, and reference numeral 90 identifies vias to the wire layers 88. Reference numeral 92 identifies the top wire layer of each of the transistors Q1, Q2, while reference numeral 94 identifies an electrode contact pad and reference numeral 96 identifies an electrode contact via. Reference numeral 98 identifies a top layer dielectric, and reference numeral 100 identifies an electrode template pad (which is preferably ˜100 nm wide). Reference numeral 102 identifies switching devices, and reference numeral 104 identifies shallow trench isolation (STI). As illustrated by reference numeral 106, the number of layers of wires>2.

As discussed above, FIG. 4 illustrates a template pad 100 being disposed on top of the electrode pad 94. This is necessary for growing nano-tube/nano-fiber electrodes (72 and 76) at low temperatures. Carbon nano-tubes, for example, can be grown at temperatures<400° C. using plasma-enhanced CVD and either metallic catalysts (e.g., Ni, Co, Fe and alloys of these metals) or nano-porous materials such as nano-porous Si/SiO₂. The template pad 100 illustrated in FIG. 4 can be any of these materials, or a combination of them. They can be introduced and patterned using techniques such as electro-plating, wet etching, ALD, lithography and dry etching. The number and position of these catalysts will define the number and locations of the nano-tubes/nano-fibers.

To maintain good detection spatial resolution, it is necessary that the nano-tubes/nano-fibers 110 be aligned normal to the contact pads 94. This can be achieved by growing these materials in plasma enhanced CVD, external electric fields or a combination of both. The length of the electrodes can be adjusted by controlling the growth time. The total number of electrodes 110 in each cluster 72, 76 can be controlled by the number of catalytic sites, and in any case will be more than 1.

An anchorage layer 80 for the nano-tube/nano-fiber electrodes 72, 76 and above the top dielectric layer 98 is needed to enhance the mechanical stability of, as well as to prevent electrical interference between, the electrodes 72, 76. This layer can be dielectrics such as SiO₂ or other materials that have good bio-compatibility, e.g., polyimide. It can be deposited on the devices after growing the electrodes 72, 76 using various film deposition techniques, e.g., CVD, PVD and ALD, so that both the electrodes 72, 76 and the top dielectrics 80, 98 are either partially or entirely covered. It can also involve planarization using techniques such as CMP, dry etching and wet etching. The materials above and around the electrodes can be removed preferentially using a combination of ion bombardment to selectively damage the structure of the materials at the selected locations followed by wet removal of the damaged material. They can also be removed through electrochemical methods, using the nano-tube/nano-fiber electrodes as electrodes for this purpose, or another selective material removal technique. The final profile of the anchorage layer around the electrodes may not be flat.

Advantages of an embodiment of the present can include, but may not be limited to:

Good bio-compatibility: Carbon is known to have excellent bio-compatibility. The use of carbon nano-tube/nano-fiber electrodes will hence provide good bio-compatibility for the devices.

Good spatial resolution: The arrangement of the electrodes in small clusters and groups ensures that activity maps can be generated from small cells, large cells and networks of cells.

Good signal-to-noise ratio: The incorporation of sensing transistors in the vicinity of the electrodes eliminates the need for long wires and minimizes parasitics. This will result in low noise levels.

Good configuration versatility: With the flexibility of wiring and electrode location manipulation, the chips will have high degree of configuration versatility, i.e., similar to Rapid Chip.

Good electrode reliability: Nano-tube/nano-fiber electrodes are known to be robust electrodes with respect to their chemical, mechanical and electrical properties. In addition, the present design uses more than one nano-tube/nano-fiber for each nano-tube/nano-fiber cluster so that back electrodes are always available in each cluster. Both of these factors ensure a good reliability of the electrodes.

New signal extraction mechanism: The differential signal extraction mode will introduce another dimension in recording cell activities and has never been proposed before.

Low power consumption: The use of low supply voltage as well as the integrated, single-chip approach of the system opens the possibility of low power consumption by the device, which in turn has important implications for certain applications.

While embodiments of the present invention are shown and described, it is envisioned that those skilled in the art may devise various modifications of the present invention without departing from the spirit and scope of the appended claims.

What is claimed is:

1. A single-chip nano-bio-sensor comprising: electronic devices on a chip; and nano-tube/nano-fiber electrodes on the chip and connected to the electronic devices, said nano-tube/nano-fiber electrodes configured to monitor cell activity and provide signals to said electronic devices, said electronic devices configured to process the signals which are received from said nano-tube/nano-fiber electrodes.

2. A single-chip nano-bio-sensor as recited in claim 1, wherein at least one of the electronic devices is configured to communicate information about the cell activity which was monitored by the nano-tube/nano-fiber electrodes, to a destination outside the chip.

3. A single-chip nano-bio-sensor as recited in claim 1, wherein said electronic devices comprise a signal acquisition module on which the nano-tube/nano-fiber electrodes are disposed.

4. A single-chip nano-bio-sensor as recited in claim 3, wherein said electronic devices further comprise reference sensors, an amplifier and a multiplexer, wherein the signal acquisition module and reference sensors are configured to provide signals to the amplifier, and the amplifier is configured to provide amplified signals to the multiplexer.

5. A single-chip nano-bio-sensor as recited in claim 4, wherein said electronic devices further comprise a signal processing unit and a communication module, wherein the multiplexer is configured to process signals received from the amplifier and provide signals to the signal processing unit which further processes the signals and provides signals to the communication module, wherein said communication module is configured to communicate information about the cell activity which was monitored by the nano-tube/nano-fiber electrodes, to a destination outside the chip.

6. A single-chip nano-bio-sensor as recited in claim 5, further comprising a power supply connected to said signal acquisition module, amplifier, reference sensors, multiplexer, signal processing unit and communication module.

7. A single-chip nano-bio-sensor as recited in claim 6, further comprising ESD protection circuitry connected to said signal acquisition module, amplifier, reference sensors, multiplexer, signal processing unit and communication module.

8. A single-chip nano-bio-sensor as recited in claim 3, wherein said signal acquisition module comprises threshold signal determining, clocking, switching, signal routing and amplification circuitries between electrode pads and under an electrode layer.

9. A single-chip nano-bio-sensor as recited in claim 3, wherein said signal acquisition module comprises groups of nano-tube/nano-fiber electrodes and signal routing wirings under the electrodes.

10. A single-chip nano-bio-sensor as recited in claim 9, wherein each nano-tube/nano-fiber electrode comprises an electrode cluster which is disposed over, and connected to, a transistor.

11. A single-chip nano-bio-sensor as recited in claim 1, wherein each nano-tube/nano-fiber electrode comprises an electrode cluster which is disposed over, and connected to, a transistor.

12. A single-chip nano-bio-sensor as recited in claim 11, wherein each electrode cluster extends from an electrode contact pad, through an electrode anchorage layer.

13. A single-chip nano-bio-sensor as recited in claim 2, wherein said electronic devices comprise a signal acquisition module on which the nano-tube/nano-fiber electrodes are disposed.

14. A single-chip nano-bio-sensor as recited in claim 13, wherein said electronic devices further comprise reference sensors, an amplifier and a multiplexer, wherein the signal acquisition module and reference sensors are configured to provide signals to the amplifier, and the amplifier is configured to provide amplified signals to the multiplexer.

15. A single-chip nano-bio-sensor as recited in claim 14, wherein said electronic devices further comprise a signal processing unit and a communication module, wherein the multiplexer is configured to process signals received from the amplifier and provide signals to the signal processing unit which further processes the signals and provides signals to the communication module, wherein said communication module is configured to communicate information about the cell activity which was monitored by the nano-tube/nano-fiber electrodes, to a destination outside the chip.

16. A single-chip nano-bio-sensor as recited in claim 15, further comprising a power supply connected to said signal acquisition module, amplifier, reference sensors, multiplexer, signal processing unit and communication module.

17. A single-chip nano-bio-sensor as recited in claim 13, wherein said signal acquisition module comprises signal routing and amplification circuitries between electrode pads and under an electrode layer.

18. A single-chip nano-bio-sensor as recited in claim 2, wherein each nano-tube/nano-fiber electrode comprises an electrode cluster which is disposed over, and connected to, a transistor.

19. A single-chip nano-bio-sensor as recited in claim 18, wherein each electrode cluster extends from an electrode contact pad, through an electrode anchorage layer.
